(19) United States
(12) Patent Application Publication
(10) Pub. No.: US 2021/0287798 A1
(43) Pub. Date: Sep. 16, 2021

(54) SYSTEMS AND METHODS FOR NON-INVASIVE VIRUS SYMPTOM DETECTION

(71) Applicant: SYMPTOMSENSE, LLC, Ronkonkoma, NY (US)

(72) Inventors: Derek Peterson, South Setauket, NY (US); Mohammed Elbadry, East Setauket, NY (US); Lenworth Anderson, Bayside, NY (US); Bilal Muhammad, West Babylon, NY (US); Asheik Hussain, Ozone Park, NY (US)

(21) Appl. No.: 17/141,448

(22) Filed: Jan. 5, 2021

Related U.S. Application Data
(60) Provisional application No. 62/989,583, filed on Mar. 13, 2020.
(60) Provisional application No. 63/027,099, filed on May 19, 2020.

(51) Int. Cl. G16H 50/20; G16H 40/67; G16H 50/70; G16H 50/30; G16H 15/00; G16H 50/80; G16H 10/60; G06N 3/04; A61B 5/05; A61B 5/00; A61B 5/01; G01J 5/00

(52) U.S. Cl. CPC G16H 50/20; G06N 3/08; G16H 50/70; G16H 50/30; G16H 15/00; G16H 50/80; G16H 10/60; G06N 3/0454; A61B 5/05; A61B 5/0059; A61B 5/015; A61B 5/0035; A61B 5/7267; A61B 5/7275; A61B 5/743; G01J 5/0025; G16H 40/67

(57) ABSTRACT

Systems for symptom detection include a sensor configured to sense a signal indicative of at least one vital sign of a user, a display, a processor, and a memory. The memory has stored thereon instructions which, when executed by the processor, cause the system to determine at least one vital sign of the user based on the sensed signal, determine a wellness and/or health condition of the user based on the at least one vital sign, and display on the display at least one of information or indicia indicative of the determined wellness or health condition.

### CROSS-REFERENCE TO RELATED APPLICATIONS

This application claims the benefit of and priority to U.S. Provisional Patent Application Ser. No. 62/989,583, filed on Mar. 13, 2020, and U.S. Provisional Patent Application Ser. No. 63/027,099, filed on May 19, 2020, the entire contents of which are incorporated by reference herein.

### TECHNICAL FIELD

The present disclosure relates to systems and methods for the detection of symptoms of diseases and/or health conditions. More particularly, the present disclosure relates to systems and methods for the detection of symptoms of diseases and/or health conditions to prevent or slow down further diseases and/or health conditions spread.

### BACKGROUND

Viruses such as influenza and more recently COVID-19 are spread easily from person to person, on surfaces, and through the air. The best way to avoid illness is to avoid being exposed to the virus. However, this is not always possible in public settings such as at work, at school, or at a sporting game.

Further, non-invasive diseases and/or health conditions symptom detection systems are needed to notify interested personnel or individuals of the potential of an infected person that passes-through gateways to a building through spatial and temporal constraints. Thus, developments in efficiently and quickly detecting non-invasive diseases and/or health conditions symptom detection are needed.

### SUMMARY

This disclosure relates to notification systems and methods for the detection of symptoms of diseases and/or health conditions to prevent or slow down further diseases and/or health conditions spread. The disclosed systems and methods focus on obtaining and analyzing at least three vital signs with high accuracy through passive non-invasive readings: surface temperature, heart rate, and chest displacement. Patients suffering from virus infections tend to show the following symptoms: shortness of breath, chilliness, sneezing, nasal congestion, cough, and/or elevated body temperature. The disclosed technology leverages non-invasive vital signs readings to detect these symptoms resulting in detecting infected persons or persons who are not healthy.

In accordance with aspects of the present disclosure, a system for symptom detection includes a sensor configured to sense a signal indicative of at least one vital sign of a user, a display, a processor, and a memory. The memory has stored thereon instructions which, when executed by the processor, cause the system to determine at least one vital sign of the user based on the sensed signal, determine a wellness or health condition of the user based on the at least one vital sign, and display on the display information or indicia indicative of the determined wellness or health condition. The determined wellness or health condition can be a negative or positive wellness or health condition. The wellness or health condition can be determined whether it is positive or negative based on whether the at least one vital sign is within a normal range (positive) or outside the normal range (negative).

In various aspects of the notification system, the signal may include a mm-wave signal. The sensor may include a mm-wave sensor.

In various aspects of the notification system, the instructions, when executed by the processor, may further cause the system to: capture the mm-wave signal by the mm-wave sensor, input the captured mm-wave signal into a vital sign model, and predict a first vital sign score based on the at least one vital sign model. The vital sign model may include a first machine learning network. The first vital sign score may be based on a characteristic of the sensed mm-wave signal, including a frequency response of the sensed mm-wave signal and/or an absorption of the mm-wave signal by the user. Determining the symptom of the disease and/or health condition may be further based on the predicted first vital sign score.

In various aspects of the notification system, the vital sign sensed by the mm-wave sensor may include at least one of an elevated heart rate, a cough, a lung congestion, and/or a respiration.

In various aspects of the notification system, the system may further include an optical sensor configured to sense an optical signal and/or a thermal imaging sensor configured for non-contact measurement of a body temperature of the user.

In various aspects of the notification system, the instructions, when executed by the processor, may further cause the system to: capture a thermal imaging signal by the thermal imaging sensor, determine the body temperature based on the thermal imaging signal, and predict a second vital sign score, by a second machine learning network, based on the body temperature.

In various aspects of the notification system, the instructions, when executed by the processor, may further cause the system to capture an optical signal, by the optical sensor, input the captured optical signal into at least one second vital sign model, the at least one second vital sign model including a third machine learning network, and predict a third vital sign score based on the at least one second vital sign model.

In various aspects of the notification system, the predicted third vital sign may be based on the optical signal.

In various aspects of the notification system, the instructions, when executed by the processor, may further cause the system to display a graph over time of a vital sign history. The vital sign history may be based on storing a value of the vital sign(s) over a predetermined period of time.

In aspects, the notification system may include means for detecting metal objects and plastic explosives.

In various aspects of the notification system, the instructions, when executed by the processor, may further cause the system to: store real-time sensor data, geographic data indicating a location of the system, and time data associated with the real-time sensor data; and generate a report showing a graphical representation of a location and a time of the results of the determined wellness and/or health condition based on the geographic data indicating a location of the system, and time data associated with the real-time sensor data.

In accordance with aspects of the present disclosure, a system for symptom detection includes a sensor configured to sense a signal indicative of at least one vital sign of a user, a display, a processor, and a memory. The memory has stored thereon instructions which, when executed by the processor, cause the system to determine a vital sign of the user based on the sensed signal, determine a symptom of a disease and/or health condition based on the vital sign, predict the existence of a suspected disease and/or health condition based on the symptom, and display on the display the results of the prediction of the suspected disease and/or health condition.

In various aspects of the notification system, the signal may include a mm-wave signal. The sensor may include a mm-wave sensor.

In various aspects of the notification system, the instructions, when executed by the processor, may further cause the system to: capture the mm-wave signal by the mm-wave sensor, input the captured mm-wave signal into a vital sign model, and predict a first vital sign score based on the at least one vital sign model. The vital sign model may include a first machine learning network. The first vital sign score may be based on a characteristic of the sensed mm-wave signal, including a frequency response of the sensed mm-wave signal and/or an absorption of the mm-wave signal by the user. Determining the symptom of the disease and/or health condition may be further based on the predicted first vital sign score.

In various aspects of the notification system, the vital sign sensed by the mm-wave sensor may include at least one of an elevated heart rate, a cough, a lung congestion, and/or a respiration.

In various aspects of the notification system, the system may further include an optical sensor configured to sense an optical signal and/or a thermal imaging sensor configured for non-contact measurement of a body temperature of the user.

In various aspects of the notification system, the instructions, when executed by the processor, may further cause the system to: capture a thermal imaging signal by the thermal imaging sensor, determine the body temperature based on the thermal imaging signal, and predict a second vital sign score, by a second machine learning network, based on the body temperature.

In various aspects of the notification system, the instructions, when executed by the processor, may further cause the system to capture an optical signal, by the optical sensor, input the captured optical signal into at least one second vital sign model, the at least one second vital sign model including a third machine learning network, and predict a third vital sign score based on the at least one second vital sign model.

In various aspects of the notification system, the predicted third vital sign may be based on the optical signal.

In various aspects of the notification system, the instructions, when executed by the processor, may further cause the system to display a graph over time of a vital sign history. The vital sign history may be based on storing a value of the vital sign(s) over a predetermined period of time.

In aspects, the notification system may include means for detecting metal objects and plastic explosives.

In various aspects of the notification system, the instructions, when executed by the processor, may further cause the system to: store real-time sensor data, geographic data indicating a location of the system, and time data associated with the real-time sensor data; and generate a report showing a graphical representation of a location and a time of the results of the prediction of the suspected disease and/or health condition based on the geographic data indicating a location of the system, and time data associated with the real-time sensor data.

In accordance with aspects of the present disclosure, a computer-implemented method for symptom detection, includes: determining at least one vital sign of a user based on a signal sensed by a sensor configured to sense a signal indicative of at least one vital sign of the user, determining a symptom of a disease and/or health condition based on the at least one vital sign, predicting an existence of a suspected disease and/or health condition based on the symptom, and displaying on a display the results of the prediction of the suspected disease and/or health condition.

In various aspects of the computer-implemented method, the signal may include a mm-wave signal. The sensor may include a mm-wave sensor.

In various aspects of the computer-implemented method, the method may further include capturing the mm-wave signal, by the mm-wave sensor, inputting the captured mm-wave signal into at least one vital sign model, and predicting a first vital sign score based on the at least one vital sign model. The at least one vital sign model may include a first machine learning network. The first vital sign score may be based on a characteristic of the sensed mm-wave signal, including a frequency response of the sensed mm-wave signal and/or an absorption of the mm-wave signal by the user. Determining the symptom of the disease and/or health condition may be further based on the predicted first vital sign score.

In various aspects of the computer-implemented method, the at least one vital sign sensed by the mm-wave sensor may include at least one of an elevated heart rate, a cough, a lung congestion, and/or respiration.

In various aspects of the computer-implemented method, the method may further include sensing an optical signal by an optical sensor, and/or a body temperature of the user by a non-contact thermal imaging sensor.

In various aspects of the computer-implemented method, the method may further include determining the body temperature based on the thermal imaging signal and predicting a second vital sign score, by a second machine learning network, based on the body temperature.

In various aspects of the computer-implemented method, the method may further include capturing an optical signal, by the optical sensor, inputting the captured optical signal into at least one second vital sign model, the at least one second vital sign model including a third machine learning network, and predicting a third vital sign score based on the at least one second vital sign model.

In various aspects of the computer-implemented method, the determined at least one vital sign may be based on the optical signal.

In various aspects of the computer-implemented method, the first machine learning network may include a convolutional neural network.

In various aspects of the computer-implemented method, the method may further include detecting at least one of a metal object or a plastic explosive based on the captured mm-wave signal.

In accordance with aspects of the present disclosure, a non-transitory computer-readable medium storing instructions which, when executed by a processor, cause the processor to perform a method for symptom detection. The method includes: determining at least one vital sign of a user based on a signal sensed by a sensor configured to sense a signal indicative of at least one vital sign of the user, determining a symptom of a disease and/or health condition based on the at least one vital sign, predicting an existence of a suspected disease and/or health condition based on the symptom, and displaying on a display the results of the prediction of the suspected disease and/or health condition.

Further details and aspects of exemplary aspects of the present disclosure are described in more detail below with reference to the appended figures.

### BRIEF DESCRIPTION OF THE DRAWINGS

A better understanding of the features and advantages of the disclosed technology will be obtained by reference to the following detailed description that sets forth illustrative aspects, in which the principles of the technology are utilized, and the accompanying figures of which:

FIG. 1 is a block diagram of a system for the detection of symptoms of diseases and/or health conditions through millimeter wave (mm-wave), in accordance with aspects of the present disclosure,

FIG. 2 is a functional block diagram of the system of FIG. 1 in accordance with aspects of the present disclosure,

FIG. 3 is a functional block diagram of a computing device in accordance with aspects of the present disclosure,

FIG. 4 is a block diagram illustrating a machine learning network in accordance with aspects of the present disclosure,

FIG. 5 is a functional block diagram of the system of FIG. 1 in accordance with aspects of the present disclosure,

FIG. 6 is a functional block diagram of a Strengths, Problems, Opportunities, and Threats (SPOT) matrix in accordance with aspects of the present disclosure, and

FIG. 7 is a flow diagram showing a method for symptom detection in accordance with aspects of the present disclosure.

### DETAILED DESCRIPTION

This disclosure relates to notification systems and methods for the detection of symptoms of diseases and/or health conditions to prevent or slow down further disease spread.

Although the present disclosure will be described in terms of specific aspects, it will be readily apparent to those skilled in this art that various modifications, rearrangements, and substitutions may be made without departing from the spirit of the present disclosure. The scope of the present disclosure is defined by the claims appended hereto.

For purposes of promoting an understanding of the principles of the present disclosure, reference will now be made to exemplary aspects illustrated in the figures, and specific language will be used to describe the same. It will nevertheless be understood that no limitation of the scope of the present disclosure is thereby intended. Any alterations and further modifications of the inventive features illustrated herein, and any additional applications of the principles of the present disclosure as illustrated herein, which would occur to one skilled in the relevant art and having possession of this disclosure, are to be considered within the scope of the present disclosure.

The disclosed detection systems and methods detect symptoms of diseases and/or health conditions to prevent or slow down further disease spread. The disclosed systems and methods include an artificial intelligence component that leverages various machine learning networks (e.g., convolutional neural networks and/or long-term short memory networks) to detect symptoms of a viral disease. The machine learning networks detect people with one or more symptoms of a viral disease that enter premises within a duration and will flag all personnel who are showing symptoms accordingly.

The term “user,” as used herein, includes a person or an animal. The term “wellness condition,” as used in herein, includes an indication that a user of the system does not have the symptoms of a suspected disease and/or health condition. The term “negative wellness condition,” as used herein, includes an indication that a user of the system has the symptoms of a suspected disease and/or health condition.

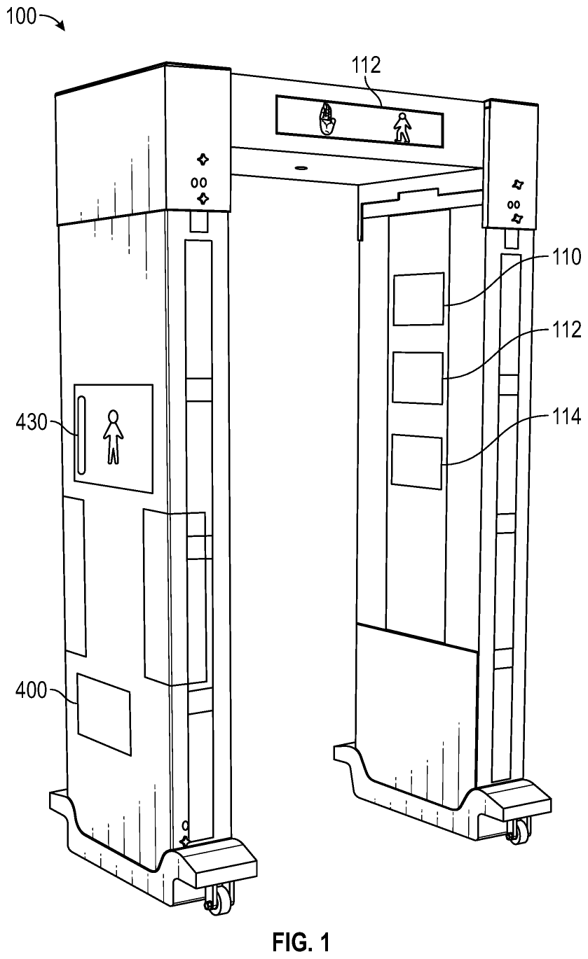

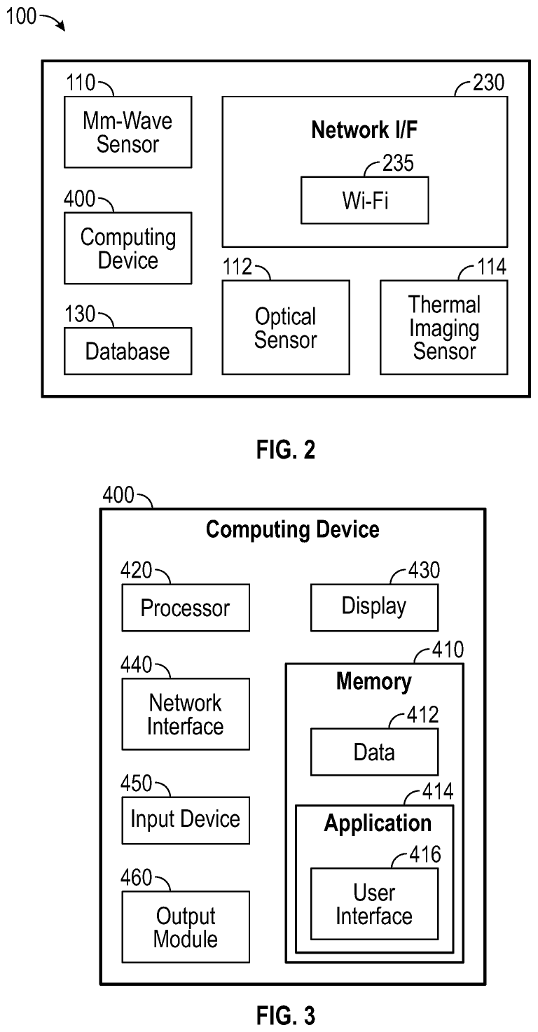

FIGS. 1 and 2 illustrate a detection system 100 for spatial and temporal symptom detection according to aspects of the present disclosure. The detection system 100 includes a mm-wave sensor 110, an optical sensor 112, a thermal imaging sensor 114, a computing device 400 for processing mm-wave sensor signals, a network interface 230, and a database 130.

The mm-wave sensor 110 is configured to detect parameters indicative of the vital signs of a user. The vital sign sensed by the mm-wave sensor includes for example, but not limited to an elevated heart rate, a cough, lung congestion, and/or respiration.

Millimeter wave sensors provide a means of examination of structures through controlled electromagnetic interactions. Both metallic and nonmetallic structures reflect and scatter electromagnetic waves striking the outer surfaces. Nonmetallic, i.e., dielectric, materials allow for electromagnetic waves to penetrate the surface and scatter or reflect off of subsurface objects and features. By measuring surface and subsurface reflectivity and scattering by the controlled launching and receiving of electromagnetic waves provides information that can indicate surface and subsurface feature geometry, material properties, and overall structural condition. Millimeter waves can be effective for vital sign measurements on personnel because the waves readily pass through most clothing materials and reflect from the body. These reflected waves can be focused by an imaging system that, for example, will analyze for accurate estimation of breathing and heart rates. Monitoring vital signs such as breathing rate and heart rate can provide crucial insights in a human's well-being and can detect a wide range of medical problems.

It is contemplated that both active and passive mm-wave imaging systems may be used in the disclosed systems and methods. Active imaging systems primarily image the reflectivity of the person/scene. Passive systems measure the thermal (e.g., black-body) emission from the scene, which will include thermal emission from the environment that is reflected by objects in the scene (including the person).

Dielectric objects, including the human body, will all produce reflections based on the Fresnel reflection at each air-dielectric or dielectric-dielectric interface. Additionally, these reflections will be altered by the shape, texture, and orientation of the surfaces. One of skill in the art is familiar with how to implement a mm-wave sensor to capture a mm-wave image.

The optical sensor 112 is configured to sense an optical signal by shining light (e.g., from a laser) into the skin of a user. Based on the sensed optical signal, the detection system 100 may be able to, for example, determine a user's oxygen level, pulse, and/or detect sweat of the user. Different amounts of this light are absorbed by blood and the surrounding tissue. The light that is not absorbed is reflected to the sensor. For example, absorption measurements with different wavelengths are used to determine the pulse rate, sweat, and/or the saturation level of oxygen in the blood. One of skill in the art is familiar with how to implement an optical sensor to capture an optical signal.

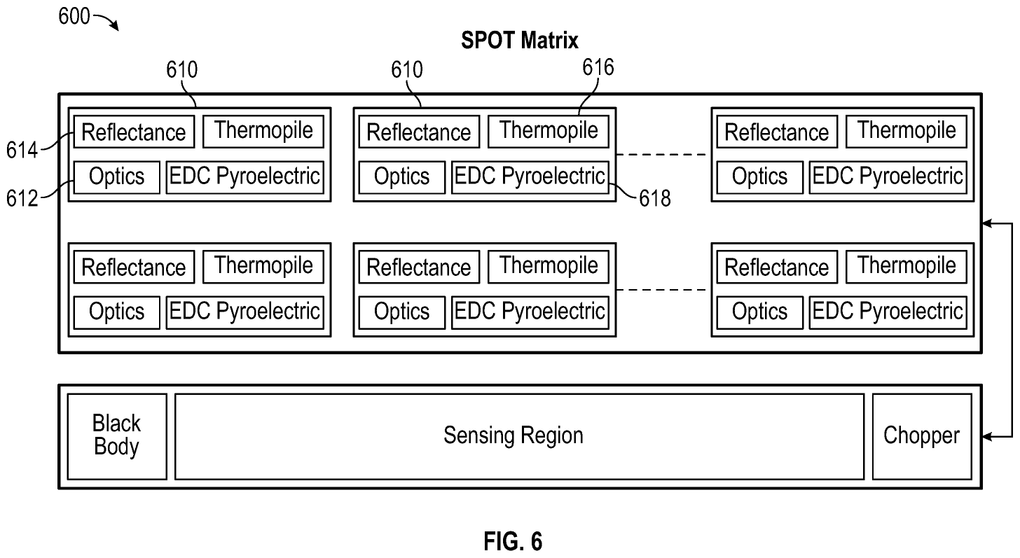

The thermal imaging sensor 114 is configured for non-contact measurement of a body temperature of the user. The thermal imaging sensor may include a Strengths, Problems, Opportunities, and Threats (SPOT) matrix sensor 600 (see FIG. 6).

The database 130 may include historical data, which is time-series and location-specific data for symptoms of a viral disease for each location where the mm-wave sensor 110, the optical sensor 112, and/or thermal imaging sensor 114 has been installed. In an aspect, the computing device 400 may analyze the historical data to predict occurrences of symptom detection at the location so that appropriate actions may be proactively and expeditiously be taken at the location.

In an aspect, when the mm-wave sensor 110, the optical sensor 112, and/or thermal imaging sensor 114 transmits detected results to the computing device 400, the computing device 400 may acquire from the database 130 the profile for the location where the mm-wave sensor 110 is installed and the time when the detected results are obtained and analyzes the detected results to identify symptoms based on the base data.

In aspects, the detection system 100 can include means for detecting metal objects and plastic explosives.

Turning now to FIG. 3, a simplified block diagram is provided for a computing device 400, which can be implemented as a control server, the database 130, a message server, and/or a client-server. The computing device 400 may include a memory 410, a processor 420, a display 430, a network interface 440, an input device 450, and/or an output module 460. The memory 410 includes any non-transitory computer-readable storage media for storing data and/or software that is executable by the processor 420 and which controls the operation of the computing device 400.

In an aspect, the memory 410 may include one or more solid-state storage devices such as flash memory chips. Alternatively, or in addition to the one or more solid-state storage devices, the memory 410 may include one or more computer-readable storage media/devices connected to the processor 420 through a mass storage controller (not shown) and a communications bus (not shown). Although the description of computer-readable media contained herein refers to solid-state storage, it should be appreciated by those skilled in the art that computer-readable storage media can be any media that can be accessed by the processor 420. That is, computer-readable storage media may include non-transitory, volatile and/or non-volatile, removable and/or non-removable media implemented in any method or technology for storage of information such as computer-readable instructions, data structures, program modules, and/or other data. For example, computer-readable storage media includes RAM, ROM, EPROM, EEPROM, flash memory or other solid-state memory technology, CD-ROM, DVD, Blu-Ray or other optical storage, magnetic cassettes, magnetic tape, magnetic disk storage or other magnetic storage devices, or any other medium which can be used to store the desired information and which can be accessed by the computing device 400.

The memory 410 may store application 414 and/or data 412 (e.g., mm-wave sensor data). The application 414 may, when executed by processor 420, cause the display 430 to present the user interface 416. The processor 420 may be a general-purpose processor, a specialized graphics processing unit (GPU) configured to perform specific graphics processing tasks while freeing up the general-purpose processor to perform other tasks, and/or any number or combination of such processors. The display 430 may be touch-sensitive and/or voice-activated, enabling the display 430 to serve as both an input and output device. Alternatively, a keyboard (not shown), mouse (not shown), or other data input devices may be employed. The network interface 440 may be configured to connect to a network such as a local area network (LAN) consisting of a wired network and/or a wireless network, a wide area network (WAN), a wireless mobile network, a Bluetooth® network, and/or the internet.

For example, the computing device 400 may receive, through the network interface 440, detection results for the mm-wave sensor 110 of FIG. 1, for example, a detected symptom from the mm-wave sensor 110. The computing device 400 may receive updates to its software, for example, the application 414, via the network interface 440. It is contemplated that updates may include “over-the-air” updates. The computing device 400 may also display notifications on the display 430 that a software update is available.

The input device 450 may be any device by which a user may interact with the computing device 400, such as, for example, a mouse, keyboard, foot pedal, touch screen, and/or voice interface. The output module 460 may include any connectivity port or bus, such as, for example, parallel ports, serial ports, universal serial buses (USB), or any other similar connectivity port known to those skilled in the art. The application 414 may be one or more software programs stored in the memory 410 and executed by the processor 420 of the computing device 400. The application 414 may be installed directly on the computing device 400 or via the network interface 440. The application 414 may run natively on the computing device 400, as a web-based application, or any other format known to those skilled in the art.

In an aspect, the application 414 will be a single software program having all of the features and functionality described in the present disclosure. In other aspects, the application 414 may be two or more distinct software programs providing various parts of these features and functionality. Various software programs forming part of the application 414 may be enabled to communicate with each other and/or import and export various settings and parameters relating to the detection of symptoms of a viral disease.

The application 414 communicates with a user interface 416, which generates a user interface for presenting visual interactive features on the display 430. For example, the user interface 416 may generate a graphical user interface (GUI) and output the GUI to the display 430 to present graphical illustrations.

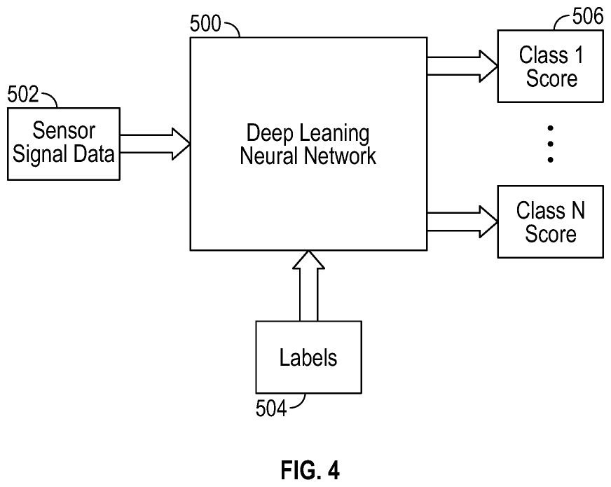

With reference to FIG. 4, a block diagram for a deep learning neural network 500 for classifying images is shown in accordance with some aspects of the disclosure. In some systems, a deep learning neural network 500 may include a convolutional neural network (CNN) and/or a recurrent neural network. Generally, a deep learning neural network includes multiple hidden layers. As explained in more detail below, the deep learning neural network 500 may leverage one or more CNNs to classify one or more images, taken by the mm-wave sensor 110 (see FIG. 2). The deep learning neural network 500 may be executed on the computing device 400 (FIG. 3). Persons skilled in the art will understand the deep learning neural network 500 and how to implement it.

In machine learning, a CNN is a class of artificial neural network (ANN), most commonly applied to analyzing visual imagery. The convolutional aspect of a CNN relates to applying matrix processing operations to localized portions of an image, and the results of those operations (which can involve dozens of different parallel and serial calculations) are sets of many features that are delivered to the next layer. A CNN typically includes convolution layers, activation function layers, and pooling (typically max pooling) layers to reduce dimensionality without losing too many features. Additional information may be included in the operations that generate these features. Providing unique information that yields features that give the neural networks information can be used to ultimately provide an aggregate way to differentiate between different data input to the neural networks.

Generally, a deep learning neural network 500 (e.g., a convolutional deep learning neural network) includes an input layer, a plurality of hidden layers, and an output layer. The input layer, the plurality of hidden layers, and the output layer are all comprised of neurons (e.g., nodes). The neurons between the various layers are interconnected via weights. Each neuron in the deep learning neural network 500 computes an output value by applying a specific function to the input values coming from the previous layer. The function that is applied to the input values is determined by a vector of weights and a bias. Learning, in the deep learning neural network, progresses by making iterative adjustments to these biases and weights. The vector of weights and the bias are called filters (e.g., kernels) and represent particular features of the input (e.g., a particular shape). The deep learning neural network 500 may output logits 506.

The deep learning neural network 500 may be trained based on labeling 504 training sensor signal data 502. For example, a sensor signal data 502 may indicate a vial sign such as pulse, and/or respiration. In some methods in accordance with this disclosure, the training may include supervised learning. The training further may include augmenting the training sensor signal data 502 to include, for example, adding noise and/or scaling of the training sensor signal data 502. Persons skilled in the art will understand training the deep learning neural network 500 and how to implement it.

In some methods in accordance with this disclosure, the deep learning neural network 500 may be used to classify sensor signal data captured by the mm-wave sensor 110 (see FIG. 2), optical sensor 112, and/or thermal sensor 114. The classification of the images may include each image being classified as a particular vital sign. For example, the image classifications may include a congestion, fever, etc. Each of the images may include a classification score. A classification score includes the outputs (e.g., logits) after applying a function such as a SoftMax to make the outputs represent probabilities.

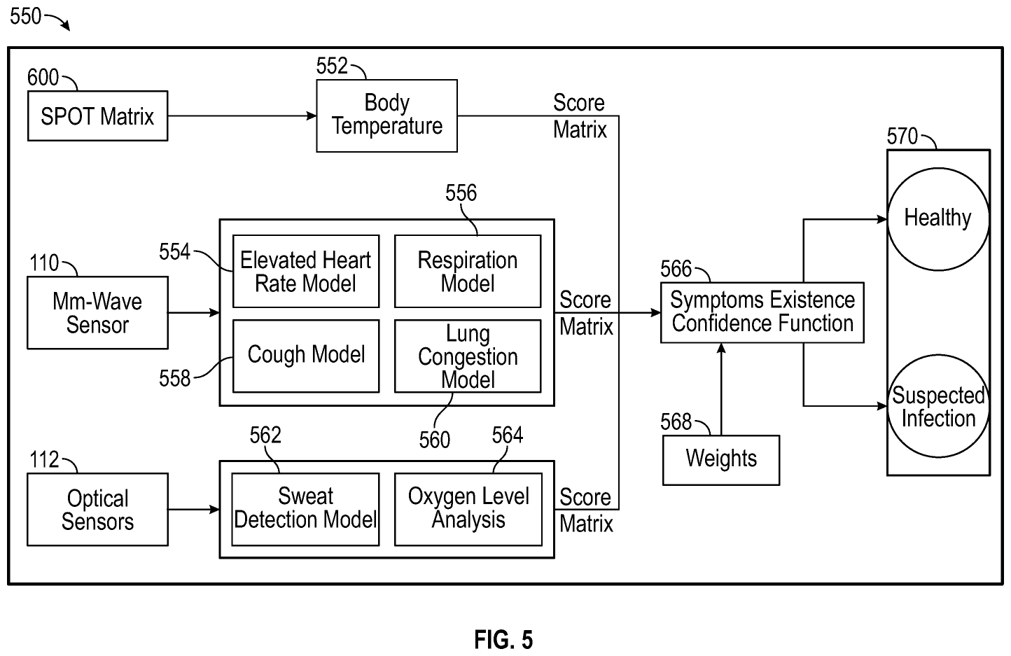

FIG. 5 is a block diagram for the detection system 100 for spatial and temporal symptom detection of FIG. 1, according to aspects of the present disclosure. In aspects, the SPOT matrix sensor 600 is configured to determine the body temperature 552 of a user. The mm-wave sensor 110 may be used to detect vital signs such as, but not limited to, respiration and pulse of a user. The vital signs may be input into a model such as an elevated heart rate model 554, a respiration model 556, a cough model 558, and/or a lung congestion model 560. The signal from the optical sensor(s) 112 may be used as inputs to a sweat detection model 562 and/or an oxygen level analysis model 564. In aspects, the results of the model(s) and/or body temperature may be part of a score matrix. The score matrix may be used by a symptom existence confidence function 566 along with various weights 568 to predict an existence of a suspected disease based on the symptom(s). The prediction may include a score, for example, “Healthy”=0.68, “Suspected Infection”=0.32. In aspects, the symptom existence confidence function 566 may include a machine learning network. An indication 570 of the prediction may be provided (e.g., “Healthy” or “Suspected Infection”).

FIG. 6 is a block diagram of an exemplary SPOT matrix sensor configured for non-contact measurement of a body temperature of the user, in accordance with aspects of the present disclosure. The SPOT matrix sensor 600 generally includes an array of sensors 610 each of which may include a thermopile 616, a pyroelectric detector 618, a reflectance sensor 614, and/or optics 612 (see FIG. 6). The thermopile 616 is an electronic device that converts thermal energy into electrical energy. The pyroelectric detector 618 is an infrared sensitive optoelectronic component which are generally used for detecting electromagnetic radiation. The reflectance sensor 614 generally includes an infrared (IR) LED that transmits IR light onto a surface and a phototransistor measures how much light is reflected back.

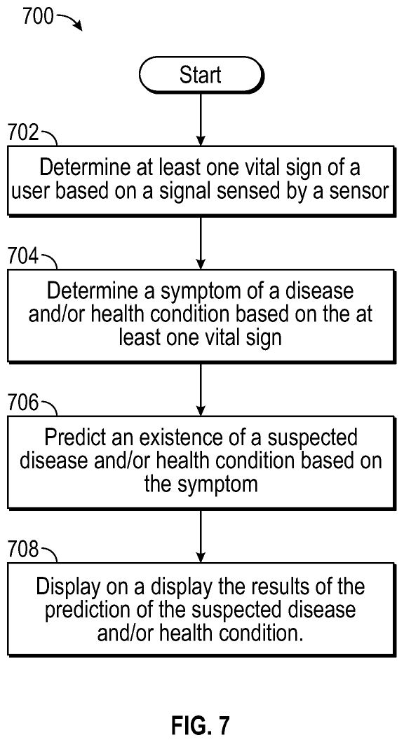

With reference to FIG. 7, a method is shown for symptom detection. Persons skilled in the art will appreciate that one or more operations of the method 700 may be performed in a different order, repeated, and/or omitted without departing from the scope of the disclosure. In various aspects, the illustrated method 700 can operate in computing device 400 (FIG. 3), in a remote device, or in another server or system. Other variations are contemplated to be within the scope of the disclosure. The operations of method 700 will be described with respect to a controller, e.g., computing device 400 (FIG. 3) of system 100 (FIG. 1), but it will be understood that the illustrated operations are applicable to other systems and components thereof as well.

The disclosed method may be executed when a person or an animal (e.g., livestock) passes through/by the system of FIG. 1.

Initially, at step 702, the method determines at least one vital sign (e.g., surface temperature, heart rate, and/or chest displacement) of a user based on a signal sensed by a sensor. More than one vital sign may be determined. In aspects, the sensor may include, but is not limited to, a mm-wave sensor, an optical sensor, and/or a thermal imaging sensor of the system of FIG. 1. For example, the vital sign sensed by the mm-wave sensor may include an elevated heart rate, a cough, a lung congestion, and/or a respiration.

In aspects, the signal may include a mm-wave signal. The method may capture the mm-wave signal, by the mm-wave sensor and input the captured mm-wave signal into a vital sign model. The vital sign model may include a machine learning network. In aspects, the method may predict a first vital sign score based on the vital sign model. The first vital sign score may be based on a characteristic of the sensed mm-wave signal, for example, a frequency response of the sensed mm-wave signal or an absorption of the mm-wave signal by the user.

In aspects, the method may detect a metal object (e.g., a weapon) and/or a plastic explosive based on the captured mm-wave signal.

The method may also sense, for example, an optical signal by the optical sensor, and/or a body temperature of the user by a thermal imaging sensor configured for non-contact measurement, of the system of FIG. 1.

In aspects, the method may capture a thermal imaging signal, by the thermal imaging sensor and determine the body temperature based on the thermal imaging signal. The method may predict a second vital sign score, by a second machine learning network (e.g., a CNN), based on the body temperature.

In aspects, the method may capture an optical signal, by the optical sensor 112 (of FIG. 1) and input the captured optical signal into at least one second vital sign model. The second vital sign model may include a machine learning network. The method may predict a third vital sign score based on the second vital sign model. In aspects, the determined vital sign may be based on the optical signal.

In various aspects, the determined vital sign(s) may be displayed on a display (e.g., the pulse of the user). The display may be a component of the system, or may be remote (e.g., on a remote station, or on a mobile device).

At step 704, the method determines a symptom of a disease and/or health condition (e.g., a virus) based on the vital sign(s). A symptom may include, but is not limited to, for example, shortness of breath, chilliness, sneezing, nasal congestion, cough, and/or elevated body temperature.

In aspects, determining the symptom of the disease and/or health condition may be further based on the predicted first vital sign score, second vital sign score, and/or third vital sign score.

In various aspects, the system may identify the symptom of the disease and/or health condition, which is in a predetermined list of symptoms of diseases. The symptom detection may be performed by a machine learning network (e.g., a convolutional neural network). For example, the machine learning network may be a CNN with six layers. In various aspects, the symptom determination may be performed locally and/or on a remote computing device.

In various aspects, the determined symptom may be displayed on a display. The display may be a component of the system, or may be remote (e.g., on a remote station, or on a mobile device).

At step 706, the method predicts an existence of a suspected disease and/or health condition based on the symptom. The suspected disease prediction may be performed by a machine learning network (e.g., a convolutional neural network). The machine learning network may be trained based on symptoms of diseases, health conditions, and/or vital signs. In various aspects, the disease prediction may be performed locally and/or on a remote computing device.

In aspects, the method may determine a wellness condition of the user based on the vital sign(s) and display on the display whether the user has a negative wellness condition. The determined wellness or health condition can be a negative or positive wellness or health condition (e.g., “healthy” or “suspected infection”). The wellness or health condition can be determined whether it is positive or negative based on whether the at least one vital sign is within a normal range (positive) or outside the normal range (negative). For example, the user may walk through the system 100, and the system 100 would detect a vital sign such as fever and display on the display that the user has a negative wellness condition.

At step 708, the method displays, on a display, the results of the prediction of the suspected disease. In aspects, the method may display a graph over time of a vital sign history. For example, the vital sign history may be based on storing a value of the vital sign(s) over a predetermined period of time. In aspects, the method may store real-time sensor data, geographic data indicating a location of the system, and time data associated with the real-time sensor data. The method may generate a report showing a graphical representation of a location and a time of the results of the prediction of the suspected disease (and/or the determined wellness or health condition) based on the geographic data indicating a location of the system and time data associated with the real-time sensor data. In aspects, the system may include more than one display where various results may be displayed. For example, the method may display the results of the prediction on one display, e.g., for viewing by an operator of the system 100, and display the symptoms on another display for viewing by the user.

In various aspects, the method may receive data from multiple sensors at different locations, for example, a building with multiple entrances with a mm-wave sensor 110 (FIG. 1) located at each entrance. The method may aggregate the data from multiple sensors.

For example, multiple people with symptoms of a disease may try and enter into multiple entrances of the building to an event (e.g., a ball game). The method would detect several people with symptoms at the various entryways and send an alert notification or display a warning. The method may predict the presence of a disease that enters premises within a duration, from one or more entrances, and will flag all personnel accordingly.

In various aspects, the method may include an alert notification to a user device estimated to be nearest to the detection sensor. The alert may be, for example, an email, a text message, or a multimedia message, among other things. The message may be sent by the mm-wave sensor 110 or sent by one or more servers, such as a client-server or a message server. In various aspects, the alert notification includes at least one of a location of the mm-wave sensor 110, a time of the detection of the sensed occurrence, a message indicating the predicted disease, symptoms of the predicted disease, vital signs of the person indicated as possibly having the predicted disease, and/or an image of the person indicated as having the predicted disease.

The aspects disclosed herein are examples of the disclosure and may be embodied in various forms. For instance, although certain aspects herein are described as separate aspects, each of the aspects herein may be combined with one or more of the other aspects herein. Specific structural and functional details disclosed herein are not to be interpreted as limiting, but as a basis for the claims and as a representative basis for teaching one skilled in the art to variously employ the present disclosure in virtually any appropriately detailed structure. Like reference, numerals may refer to similar or identical elements throughout the description of the figures.

The phrases “in an embodiment,” “in aspects,” “in various aspects,” “in some aspects,” or “in other aspects” may each refer to one or more of the same or different aspects in accordance with the present disclosure. A phrase in the form “A or B” means “(A), (B), or (A and B).” A phrase in the form “at least one of A, B, or C” means “(A), (B), (C), (A and B), (A and C), (B and C), or (A, B, and C).”

Any of the herein described methods, programs, algorithms or codes may be converted to, or expressed in, a programming language or computer program. The terms “programming language” and “computer program,” as used herein, each include any language used to specify instructions to a computer, and include (but is not limited to) the following languages and their derivatives: Assembler, Basic, Batch files, BCPL, C, C+, C++, Delphi, Fortran, Java, JavaScript, machine code, operating system command languages, Pascal, Perl, PL1, scripting languages, Visual Basic, metalanguages which themselves specify programs, and all first, second, third, fourth, fifth, or further generation computer languages. Also included are database and other data schemas, and any other meta-languages. No distinction is made between languages that are interpreted, compiled, or use both compiled and interpreted approaches. No distinction is made between compiled and source versions of a program. Thus, reference to a program, where the programming language could exist in more than one state (such as source, compiled, object, or linked) is a reference to any and all such states. Reference to a program may encompass the actual instructions and/or the intent of those instructions.

It should be understood that the foregoing description is only illustrative of the present disclosure. Various alternatives and modifications can be devised by those skilled in the art without departing from the disclosure. Accordingly, the present disclosure is intended to embrace all such alternatives, modifications and variances. The aspects described with reference to the attached figures are presented only to demonstrate certain examples of the disclosure. Other elements, steps, methods, and techniques that are insubstantially different from those described above and/or in the appended claims are also intended to be within the scope of the disclosure.

What is claimed is:

1. A system for symptom detection, comprising:
a sensor configured to sense a signal indicative of at least one vital sign of a user;
a display;
a processor; and
a memory having stored thereon instructions which, when executed by the processor, cause the system to:
determine at least one vital sign of the user based on the sensed signal;
determine at least one of a wellness or a health condition of the user based on the at least one vital sign; and
display on the display, at least one of information or indicia indicative of the determined wellness or health condition.

2. The system of claim 1, wherein the determined wellness or health condition is a negative or positive wellness or health condition.

3. The system of claim 2, wherein the signal includes a mm-wave signal, and wherein the sensor includes a mm-wave sensor.

4. The system of claim 3, wherein the instructions, when executed by the processor, further cause the system to:
capture the mm-wave signal, by the mm-wave sensor;
input the captured mm-wave signal into at least one vital sign model, the at least one vital sign model including a first machine learning network; and
predict a first vital sign score based on the at least one vital sign model,
wherein the first vital sign score is based on a characteristic of the sensed mm-wave signal, including at least one of a frequency response of the sensed mm-wave signal or an absorption of the mm-wave signal by the user, and
wherein determining the symptom of the disease or health condition is further based on the predicted first vital sign score.

5. The system of claim 4, wherein the at least one vital sign sensed by the mm-wave sensor includes at least one of an elevated heart rate, a cough, a lung congestion, or a respiration.

6. The system of claim 2, further comprising at least one of an optical sensor configured to sense an optical signal, or a thermal imaging sensor configured for non-contact measurement of a body temperature of the user.

7. The system of claim 6, wherein the instructions, when executed by the processor, further cause the system to:
capture a thermal imaging signal, by the thermal imaging sensor;
determine the body temperature based on the thermal imaging signal; and
predict a second vital sign score, by a second machine learning network, based on the body temperature.

8. The system of claim 6, wherein the instructions, when executed by the processor, further cause the system to:
capture an optical signal, by the optical sensor;
input the captured optical signal into at least one second vital sign model, the at least one second vital sign model including a third machine learning network; and
predict a third vital sign score based on the at least one second vital sign model.

9. The system of claim 8, wherein the predicted third vital sign is based on the optical signal.

10. The system of claim 2, wherein the instructions, when executed by the processor, further cause the system to:
display a graph over time of a vital sign history,
wherein the vital sign history is based on storing a value of the at least one vital sign over a predetermined period of time.

11. The system of claim 2, wherein the instructions, when executed by the processor, further cause the system to:
store real-time sensor data, geographic data indicating a location of the system, and time data associated with the real-time sensor data; and
generate a report showing a graphical representation of a location and a time of the results of the determined wellness or health condition based on the geographic data indicating a location of the system, and time data associated with the real-time sensor data.

12. A system for symptom detection, comprising:
a sensor configured to sense a signal indicative of at least one vital sign of a user;
a display;
a processor; and
a memory having stored thereon instructions which, when executed by the processor, cause the system to:
determine at least one vital sign of the user based on the sensed signal;
determine a symptom of at least one of a disease or health condition based on the at least one vital sign;
predict an existence of a suspected disease or health condition based on the symptom; and
display on the display the results of the prediction of the suspected disease or health condition.

13. The system of claim 12, wherein the signal includes a mm-wave signal, and wherein the sensor includes a mm-wave sensor.

14. The system of claim 13, wherein, the instructions, when executed by the processor, further cause the system to:
capture the mm-wave signal, by the mm-wave sensor;
input the captured mm-wave signal into at least one vital sign model, the at least one vital sign model including a first machine learning network; and
predict a first vital sign score based on the at least one vital sign model,
wherein the first vital sign score is based on a characteristic of the sensed mm-wave signal, including at least one of a frequency response of the sensed mm-wave signal or an absorption of the mm-wave signal by the user, and
wherein determining the symptom of the disease or health condition is further based on the predicted first vital sign score.

15. The system of claim 14, wherein the at least one vital sign sensed by the mm-wave sensor includes at least one of an elevated heart rate, a cough, a lung congestion, or a respiration.

16. The system of claim 12, further comprising at least one of an optical sensor configured to sense an optical signal, or a thermal imaging sensor configured for non-contact measurement of a body temperature of the user.

17. The system of claim 16, wherein the instructions, when executed by the processor, further cause the system to:
capture a thermal imaging signal, by the thermal imaging sensor;
determine the body temperature based on the thermal imaging signal; and
predict a second vital sign score, by a second machine learning network, based on the body temperature.

18. The system of claim 16, wherein the instructions, when executed by the processor, further cause the system to:
capture an optical signal, by the optical sensor;
input the captured optical signal into at least one second vital sign model, the at least one second vital sign model including a third machine learning network; and
predict a third vital sign score based on the at least one second vital sign model.

19. The system of claim 18, wherein the predicted third vital sign is based on the optical signal.

20. The system of claim 12, wherein the instructions, when executed by the processor, further cause the system to:
display a graph over time of a vital sign history,
wherein the vital sign history is based on storing a value of the at least one vital sign over a predetermined period of time.

21. The system of claim 12, wherein the instructions, when executed by the processor, further cause the system to:
store real-time sensor data, geographic data indicating a location of the system, and time data associated with the real-time sensor data; and
generate a report showing a graphical representation of a location and a time of the results of the prediction of the suspected disease or health condition based on the geographic data indicating a location of the system, and time data associated with the real-time sensor data.

22. A computer-implemented method for symptom detection, comprising:
determining at least one vital sign of a user based on a signal sensed by a sensor configured to sense a signal indicative of at least one vital sign of the user;
determining a symptom of at least one of a disease or health condition based on the at least one vital sign;
predicting an existence of a suspected disease or health condition based on the symptom; and
displaying on a display the results of the prediction of the suspected disease or health condition.

23. The computer-implemented method of claim 22, wherein the signal includes a mm-wave signal, and wherein the sensor includes a mm-wave sensor.

24. The computer-implemented method of claim 23, further comprising:
capturing the mm-wave signal, by the mm-wave sensor;
inputting the captured mm-wave signal into at least one vital sign model, the at least one vital sign model including a first machine learning network; and
predicting a first vital sign score based on the at least one vital sign model,
wherein the first vital sign score is based on a characteristic of the sensed mm-wave signal, including at least one of a frequency response of the sensed mm-wave signal or an absorption of the mm-wave signal by the user, and
wherein determining the symptom of the disease or health condition is further based on the predicted first vital sign score.

25. The computer-implemented method of claim 24, wherein the at least one vital sign sensed by the mm-wave sensor includes at least one of an elevated heart rate, a cough, a lung congestion, or respiration.

26. The computer-implemented method of claim 22, further comprising sensing at least one of an optical signal by an optical sensor, or a body temperature of the user by a non-contact thermal imaging sensor.

27. The computer-implemented method of claim 26, further comprising;
determining the body temperature based on the thermal imaging signal; and
predicting a second vital sign score, by a second machine learning network, based on the body temperature.

28. The computer-implemented method of claim 26, further comprising:
capturing an optical signal, by the optical sensor;
inputting the captured optical signal into at least one second vital sign model, the at least one second vital sign model including a third machine learning network; and
predicting a third vital sign score based on the at least one second vital sign model.

29. The computer-implemented method of claim 28, wherein the determined at least one vital sign is based on the optical signal.

30. The computer-implemented method of claim 24, wherein the first machine learning network includes a convolutional neural network.

31. The computer-implemented method of claim 24, further comprising:
detecting at least one of a metal object or a plastic explosive based on the captured mm-wave signal.

32. A non-transitory computer-readable medium storing instructions which, when executed by a processor, cause the processor to perform a method for symptom detection, the method comprising:
determining at least one vital sign of a user based on a signal sensed by a sensor configured to sense a signal indicative of at least one vital sign of the user;
determining a symptom of at least one of a disease or health condition based on the at least one vital sign;
predicting an existence of a suspected disease based on the symptom; and
displaying on a display the results of the prediction of the suspected disease or health condition.
